(19) United States
(12) Patent Application Publication
(10) Pub. No.: US 2014/0010883 A1
(43) Pub. Date: Jan. 9, 2014

(54) CONTROLLED RELEASE PHARMACEUTICAL COMPOSITIONS OF SELECTIVE SEROTONIN REUPTAKE INHIBITOR

(71) Applicants: Harshal Jahagirdar, Pune (IN); Ganesh Katkar, Pune (IN); Satish Kumar Dalal, Pune (IN); Shirishkumar Kulkarni, Pune (IN)

(72) Inventors: Harshal Jahagirdar, Pune (IN); Ganesh Katkar, Pune (IN); Satish Kumar Dalal, Pune (IN); Shirishkumar Kulkarni, Pune (IN)

(73) Assignee: LUPIN LIMITED, Mumbai, Maharashtra (IN)

(21) Appl. No.: 14/005,613

(22) PCT Filed: Mar. 16, 2012
(86) PCT No.: PCT/IB12/51272

§ 371 Date: Sep. 17, 2013

(30) Foreign Application Priority Data
Mar. 17, 2011 (IN) 358/KOL/2011

(51) Int. Cl. A61K 9/50; A61K 31/4525

(52) U.S. Cl. CPC A61K 9/50; A61K 31/4525
USPC 424/490; 514/321

(57) ABSTRACT

The present invention provides a controlled release pharmaceutical composition comprising selective serotonin reuptake inhibitor and a process for preparation thereof. The controlled release pharmaceutical composition comprises: a deposit-core comprising a selective serotonin reuptake inhibitor, one or more controlled release agent(s) and one or more pharmaceutically acceptable excipient(s), a support-platform applied to the deposit-core, a controlled release coating and, an immediate release coating comprising a selective serotonin reuptake inhibitor and one or more pharmaceutically acceptable excipient(s).

### FIELD OF THE INVENTION

The present invention relates to controlled release pharmaceutical compositions comprising selective serotonin reuptake inhibitor and a process for preparation thereof.

### BACKGROUND OF THE INVENTION

Selective serotonin reuptake inhibitors are useful for the treatment of psychiatric problems such as depression, Parkinson's disease, anxiety disorders, obsessive-compulsive disorders, panic disorder, post-traumatic stress disorder and numerous other conditions.

Selective serotonin reuptake inhibitors include but not limited to Fluoxetine (U.S. Pat. No. 4,314,081), Fluvoxamine (U.S. Pat. No. 4,085,225), Desvenlafaxine (U.S. Pat. No. 6,673,838) and Sertraline (U.S. Pat. No. 4,536,518).

Paroxetine is a selective serotonin reuptake inhibitor and chemically, Paroxetine is (−)-trans-3-[(1,3-benzodioxol-5-yloxy)methyl]-4-(4-fluorophenyl)piperidine as disclosed in U.S. Pat. No. 4,007,196.

Paroxetine hydrochloride hemihydrate disclosed in U.S. Pat. No. 4,721,723.

It is useful for the treatment of psychiatric problems such as depression, Parkinson's disease, anxiety disorders, obsessive-compulsive disorders, panic disorder, post-traumatic stress disorder and numerous other conditions.

Conventional dosage form of Paroxetine requires frequent dosing to maintain reasonably stable plasma concentrations. However, frequent dosing results in increased frequency of side effects such as nausea and vomiting and leads to poor patient compliance. In view of these problems controlled-release pharmaceutical composition comprising Paroxetine has been developed which results in reduced frequency of dosing, improved patient compliance and reduced frequency of side effects.

U.S. Pat. No. 4,839,177 and U.S. Pat. No. 5,422,123 disclose controlled release dosage forms consisting of a deposit core of defined geometrical form containing the active substance, polymer substances which swell on contact with aqueous liquids and polymer substances with gelling properties, and a support platform coat partially coating the deposit core wherein the support platform consisting of polymer substances which are slowly soluble and/or slowly gellable in aqueous liquids, plasticizing substances, and other adjuvants.

U.S. Pat. No. 6,548,084 discloses the bilayer enteric-coated tablet formulation of Paroxetine hydrochloride where the active layer is having a defined geometric form along with support platform. It is further disclosed that the controlled and delayed release formulation containing Paroxetine give rise to reduction in the side effects associated with the swallow tablets. The dosage forms are formulated in a manner such that release of active substance is affected predominantly in the small intestine.

Paroxetine is commercially marketed by GlaxoSmithKline in the form of hydrochloride hemihydrate salt as immediate release tablets under the trade name Paxil® and controlled release tablets under the trade name Paxil CR®.

U.S. Pat. No. 7,229,640 discloses controlled and delayed release swallow pharmaceutical composition that reduces incidences of nausea and vomiting associated with administration of Paroxetine.

US Patent Application No. 2006/0039975 discloses a controlled release dosage forms comprising release-retarding materials other than hydroxypropyl methylcellulose and also methods of wet granulating controlled release Paroxetine dosage forms.

US Patent Application No. 2005/0266082 discloses a hydrophobic matrix comprised of Paroxetine hydrochloride and a lipid component is provided, wherein the matrix also preferably contains hydrophilic polymers and methods of making such a composition by melt granulating Paroxetine HCl with a molten binder comprising a lipid component.

US Patent Application No. 2005/034954 discloses a modified release composition comprising Paroxetine, microcrystalline cellulose, at least one modified release polymer, and one more pharmaceutically acceptable inert excipients, where in the pharmaceutical composition is prepared by a wet granulation technique.

WO 2007/015270 discloses a controlled release composition comprising a) a core comprising the active ingredient, one or more controlled release polymer(s) and one or more pharmaceutically acceptable excipients; and optionally b) a coating comprising one or more controlled release polymers.

WO 2005/107715 discloses a controlled release tablet comprising a core consisting of Paroxetine, at least one rate controlling hydrophilic polymer, a diluent, a binder and a lubricant; and (ii) a coating consisting of an enteric polymer and a plasticizers.

WO 2006/123364 discloses an oral drug delivery system comprising a core comprising active ingredient, and a coating surrounding the core, the coating includes a water-insoluble cellulose derivative, preferably ethyl cellulose and a pH-dependent polymer, preferably a methacrylic acid derivative.

WO 2007/011139 discloses sustained-release tablet comprising Paroxetine hydrochloride, a highly viscous polymer, a low viscous hydroxy propyl methylcellulose and a pharmaceutically acceptable excipient.

WO 2007/028587 discloses a controlled release multiple unit dosage form comprising: (i) an inert core unit comprising ethyl cellulose and optionally one or more water-soluble or water-swellable excipients; (ii) an active layer on the surface of the inert core comprising one or more active ingredients and one or more hydrophilic polymers; (iii) and polymeric layer over the active layer, wherein the polymeric layer is effective for controlling or modifying the release of active ingredient.

WO 2007/035815 discloses a composition comprising: a) a compressed core containing a mixture comprising: Paroxetine or a salt thereof, ethylcellulose, and a hydroxypropyl methylcellulose polymer having a nominal viscosity about 5 to about 100 cP; or Paroxetine or a salt thereof and a combination of a hydroxypropyl methylcellulose polymer having a nominal viscosity about 25,000 to about 100,000 cP and a hydroxypropyl methylcellulose polymer having a nominal viscosity about 5 to about 100 cP; and b) a coating over the core comprising an acid-resistant polymer.

US Patent Application No. 2009/130206 discloses controlled release dosage form that releases 15% of Paroxetine in first 2 hours and releasing the remaining amount of Paroxetine in a controlled manner can effectively minimizes the incidence of side effects by avoiding the release of Paroxetine in the stomach.

IN 872/KOL/2007 discloses modified release pharmaceutical composition comprising a) a controlled release core comprising one or more selective serotonin reuptake inhibitor or pharmaceutically acceptable salts thereof, one or more controlled release agent(s) and pharmaceutically acceptable excipient(s), and b) one or more coatings comprising an enteric coat over the core and, c) an outer immediate release drug coating comprising at least 10% of the active agent, wherein the dosage form releases more than 10% of the active pharmaceutical ingredient within 2 hours.

While there are many compositions available to reduce side effects of conventional dosage forms, still there remains a need to develop a controlled release composition of selective serotonin reuptake inhibitor.

### OBJECTIVE OF THE INVENTION

The main object of invention is to provide controlled release pharmaceutical compositions of selective serotonin reuptake inhibitor, more particularly, the present invention relates to controlled release compositions comprising Paroxetine.

Another object of invention is a controlled release pharmaceutical composition comprises a deposit-core comprising a selective serotonin reuptake inhibitor, one or more controlled release agent(s) and one or more pharmaceutically acceptable excipient(s), a support-platform applied to the deposit-core, a controlled release coating and, an immediate release coating comprising a selective serotonin reuptake inhibitor and one or more pharmaceutically acceptable excipient(s).

Another object of invention is a controlled release pharmaceutical composition comprises a deposit-core comprising a selective serotonin reuptake inhibitor, one or more controlled release agent(s) and one or more pharmaceutically acceptable excipient(s), a support-platform applied to the deposit-core, a controlled release coating and, an immediate release coating comprising a selective serotonin reuptake inhibitor and one or more pharmaceutically acceptable excipient(s), wherein immediate release coating comprises about 16% to about 25% of total selective serotonin reuptake inhibitor and deposit-core comprises about 75% to about 84% of total selective serotonin reuptake inhibitor.

Another object of the invention is a controlled release pharmaceutical composition comprises a deposit-core comprising a selective serotonin reuptake inhibitor, one or more controlled release agent(s) and one or more pharmaceutically acceptable excipient(s), a support-platform applied to the deposit-core, a controlled release coating and, an immediate release coating comprising a selective serotonin reuptake inhibitor and one or more pharmaceutically acceptable excipient(s), wherein composition releases more than 15% of selective serotonin reuptake inhibitor in first two hours measured using USP Type II dissolution apparatus in 900 ml of 0.1 N HCl at 50 rpm.

Another object of the invention is a controlled release pharmaceutical composition comprises a deposit-core comprising a selective serotonin reuptake inhibitor, one or more controlled release agent(s) and one or more pharmaceutically acceptable excipient(s), a support-platform applied to the deposit-core, a controlled release coating and, an immediate release coating comprises about 16% to about 25% of total selective serotonin reuptake inhibitor and one or more pharmaceutically acceptable excipient(s), wherein composition releases more than 15% of selective serotonin reuptake inhibitor in first two hours measured using USP Type II dissolution apparatus in 900 ml of 0.1 N HCl at 50 rpm.

### SUMMARY OF THE INVENTION

The present invention provides controlled release pharmaceutical compositions and a process for preparation thereof. The present invention relates to a controlled release pharmaceutical composition comprises a deposit-core comprising a selective serotonin reuptake inhibitor, one or more controlled release agent(s) and one or more pharmaceutically acceptable excipient(s), a support-platform applied to the deposit-core, a controlled release coating and, an immediate release coating comprising a selective serotonin reuptake inhibitor and one or more pharmaceutically acceptable excipient(s). More particularly, the invention relates to controlled release compositions comprising Paroxetine and a process for preparation thereof.

### DETAILED DESCRIPTION OF THE INVENTION

The present invention relates to controlled release pharmaceutical compositions of selective serotonin reuptake inhibitor, more particularly, the invention relates to controlled release pharmaceutical compositions comprising Paroxetine and a process for preparation thereof.

The term, “Selective Serotonin Reuptake Inhibitor” (SSRI) includes but not limited to Sertraline, Fluoxetine, Fluvoxamine, Citalopram, Escitalopram, Desvenlafaxine and Paroxetine.

It will be understood that, for the purposes of the invention, SSRI will include all forms of these compounds and not limited to their base, pharmaceutically acceptable salt(s) or enantiomer(s) or polymorph(s) thereof.

Paroxetine used in the present invention is in the form of base, pharmaceutically acceptable salt(s) or enantiomer(s) or polymorph(s) thereof. Preferably, Paroxetine is in the form of the hydrochloride hemihydrate.

Selective Serotonin Reuptake Inhibitors used in pharmaceutical compositions of invention in an amount of which is safe, well tolerated in patients with acceptable adverse effect profiles and are those in common practice and known to person skilled in the art.

A controlled release pharmaceutical composition according to the invention comprises but is not limited to tablets (single layered tablets, multilayered tablets, mini tablets, bioadhesive tablets, caplets, matrix tablets, tablet within a tablet, mucoadhesive tablets, modified release tablets, pulsatile release tablets, and timed release tablets), pellets, beads, granules, sustained release formulations, capsules, microcapsules, tablets in capsules, microspheres, matrix formulations, microencapsulation.

In one embodiment, a controlled release pharmaceutical composition comprises: a deposit-core comprising a selective serotonin reuptake inhibitor, one or more controlled release agent(s) and one or more pharmaceutically acceptable excipient(s), a support-platform applied to the deposit-core, a controlled release coating and, an immediate release coating comprising a selective serotonin reuptake inhibitor and one or more pharmaceutically acceptable excipient(s).

In another embodiment, a controlled release pharmaceutical composition comprises: a deposit-core comprising Paroxetine, one or more controlled release agent(s) and one or more pharmaceutically acceptable excipient(s), a support-platform applied to the deposit-core, a controlled release coating and, an immediate release coating comprising Paroxetine and one or more pharmaceutically acceptable excipient(s).

The term “deposit-core” refers to core comprising the active substance, one or more controlled release agent(s) and one or more pharmaceutically acceptable excipient(s) and which has defined geometrical form.

The deposit-core is generally obtained by compressing the mixture containing the active substance, one or more controlled release agent(s) and one or more pharmaceutically acceptable excipient(s). The controlled release agent(s) in the deposit-core is hydrophilic, hydrophobic or combinations thereof.

The hydrophilic controlled release agent(s) according to invention comprises but not limited cellulose derivatives, alginic acid derivatives, polysaccharides, alkylene oxides or mixtures thereof.

Preferably, hydrophilic controlled release agent(s) comprises celluloses or their salts or derivatives thereof, hydroxyethylcellulose, hydroxypropyl cellulose, hydroxypropyl methylcellulose (hypromellose), sodium carboxymethyl cellulose, alginic acid or their salts and derivatives thereof, carbomer (Carbopol™), polyethyleneoxide, xanthan gum, guar gum, locust bean gum, poly vinyl acetate, polyvinyl alcohol, lactose.

The hydrophobic controlled release agent(s) according to the invention comprises but not limited to hydrogenated vegetable oils, polymethacrylates, ethyl cellulose or mixtures thereof.

Preferably, hydrophobic controlled release agent(s) comprises Ammonio methacrylate copolymers type A and B as described in USP, methacrylic acid copolymer type A, B and C as described in USP, Polyacrylate dispersion 30% as described in Ph. Eur., Polyvinyl acetate dispersion, ethylcellulose, cellulose acetate, cellulose propionate (lower, medium or higher molecular weight), cellulose acetate propionate, cellulose acetate butyrate, cellulose acetate phthalate, cellulose triacetate, poly(methyl methacrylate), poly(ethyl methacrylate), poly(butyl methacrylate), poly(isobutyl methacrylate), and poly(hexyl methacrylate). Poly(isodecyl methacrylate), poly(lauryl methacrylate), poly(phenyl methacrylate), poly(methyl acrylate), poly(isopropyl acrylate), poly(isobutyl actylate), poly(octadecyl acrylate), waxes such as beeswax, carnauba wax, microcrystalline wax, and ozokerite; fatty alcohols such as cetostearyl alcohol, stearyl alcohol; cetyl alcohol and myristyl alcohol; and fatty acid esters such as glyceryl monostearate, glycerol distearate; glycerol monooleate, acetylated monoglycerides, tristearin, tripalmitin, cetyl esters wax, glyceryl palmitostearate, glyceryl behenate, and hydrogenated castor oil.

The term “support-platform” refers to platform for keeping the deposit-core in place.

In another embodiment, a controlled pharmaceutical composition of invention comprises support-platform wherein support-platform is elastic or rigid.

The term “rigid support-platform” refers to support platform which do not follows changes due to hydration of the deposit-core and which flaking off before the active substance has been completely released.

The term “elastic support-platform” refers to support platform which follows changes due to hydration of the deposit-core and which remains intact until the complete release of the active substance.

Support platform comprises polymeric substances, plasticizer, binder and one or more pharmaceutically acceptable excipients, wherein plasticizing action can also be performed by the polymer substances.

The polymer substances comprises cellulose derivatives like hydroxypropylmethylcellulose having a molecular weight of between 4,000 and 2,000,000, high molecular weight carboxyvinylpolymers, polyvinylalcohols, polyvinylpyrrolidone (PVP), scleroglucans, acrylates, methacrylates, hydroxypropylcellulose, sodium carboxymethylcellulose, acrylates, celluloses, ethylcellulose, cellulose acetate-propionate, polyethylenes and methacrylates and copolymers of acrylic acid, polyvinylalcohols.

The plasticizer able to provide elasticity comprises polyethylene glycols, castor oil, hydrogenated castor oil, ethyl phthalate, butyl phthalate, and natural, synthetic and semisynthetic glycerides.

Support-platform can be obtained by compressing polymeric substances, plasticizer, binder and one or more pharmaceutically acceptable excipients onto the deposit-core, by immersing the deposit-core in a solution of polymeric substances, plasticizer, binder and one or more pharmaceutically acceptable excipients in normal organic solvents, or by spraying the solutions of polymeric substances, plasticizer, binder and one or more pharmaceutically acceptable excipients.

The support platform applied to deposit-core to partially or completely cover its surface.

In one embodiment, a controlled release pharmaceutical composition comprises a deposit-core comprising a selective serotonin reuptake inhibitor, one or more controlled release agent(s) and one or more pharmaceutically acceptable excipient(s), a support-platform applied to the deposit-core, a controlled release coating and, an immediate release coating comprising a selective serotonin reuptake inhibitor and one or more pharmaceutically acceptable excipient(s), wherein selective serotonin reuptake inhibitor present in deposit-core and immediate release layer are same or different.

In another embodiment, a controlled release pharmaceutical composition comprises a deposit-core comprising a selective serotonin reuptake inhibitor, one or more controlled release agent(s) and one or more pharmaceutically acceptable excipient(s), a support-platform applied to the deposit-core, a controlled release coating and, an immediate release coating comprising a selective serotonin reuptake inhibitor and one or more pharmaceutically acceptable excipient(s), wherein immediate release coating comprises about 16% to about 25% of total selective serotonin reuptake inhibitor and deposit-core comprises about 75% to about 84% of total selective serotonin reuptake inhibitor.

In one embodiment, an immediate release coating of invention comprises about 16% to about 25% of total selective serotonin reuptake inhibitor, preferably about 18% to about 25% of total selective serotonin reuptake inhibitor, more preferably about 20% of total selective serotonin reuptake inhibitor.

The immediate release coating of invention comprising a selective serotonin reuptake inhibitor and one or more pharmaceutically acceptable excipient(s).

In another embodiment, a controlled release pharmaceutical composition comprises a deposit-core comprising Paroxetine, one or more controlled release agent(s) and one or more pharmaceutically acceptable excipient(s), a support-platform applied to the deposit-core, a controlled release coating and, an immediate release coating comprising Paroxetine and one or more pharmaceutically acceptable excipient(s), wherein immediate release coating comprises about 16% to about 25% of total Paroxetine and deposit-core comprises about 75% to about 84% of total Paroxetine.

In one embodiment, an immediate release coating of invention comprises about 16% to about 25% of total Paroxetine, preferably about 18% to about 25% of total selective Paroxetine, more preferably about 20% of total Paroxetine.

In another embodiment, a controlled release pharmaceutical composition comprises a deposit-core comprising a selective serotonin reuptake inhibitor, one or more controlled release agent(s) and one or more pharmaceutically acceptable excipient(s), a support-platform applied to the deposit-core, a controlled release coating and, an immediate release coating comprising a selective serotonin reuptake inhibitor and one or more pharmaceutically acceptable excipient(s), wherein composition releases more than 15% of selective serotonin reuptake inhibitor in first two hours measured using USP Type II dissolution apparatus in 900 ml of 0.1 N HCl at 50 rpm.

In another embodiment, a controlled release pharmaceutical composition comprises a deposit-core comprising a selective serotonin reuptake inhibitor, one or more controlled release agent(s) and one or more pharmaceutically acceptable excipient(s), a support-platform applied to the deposit-core, a controlled release coating and, an immediate release coating comprises about 16% to about 25% of total selective serotonin reuptake inhibitor and one or more pharmaceutically acceptable excipient(s), wherein composition releases more than 15% of selective serotonin reuptake inhibitor in first two hours measured using USP Type II dissolution apparatus in 900 ml of 0.1 N HCl at 50 rpm.

In another embodiment, a stable controlled release pharmaceutical composition comprises a deposit-core comprising a selective serotonin reuptake inhibitor, one or more controlled release agent(s) and one or more pharmaceutically acceptable excipient(s), a support-platform applied to the deposit-core, a controlled release coating and, an immediate release coating comprising a selective serotonin reuptake inhibitor and one or more pharmaceutically acceptable excipient(s).

In another embodiment, a stable controlled release pharmaceutical composition comprises a deposit-core comprising a selective serotonin reuptake inhibitor, one or more controlled release agent(s) and one or more pharmaceutically acceptable excipient(s), a support-platform applied to the deposit-core, a controlled release coating and, an immediate release coating comprising a selective serotonin reuptake inhibitor and one or more pharmaceutically acceptable excipient(s), wherein immediate release coating comprises about 16% to about 25% of total selective serotonin reuptake inhibitor and deposit-core comprises about 75% to about 84% of total selective serotonin reuptake inhibitor.

In another embodiment, a stable controlled release pharmaceutical composition comprises a deposit-core comprising a selective serotonin reuptake inhibitor, one or more controlled release agent(s) and one or more pharmaceutically acceptable excipient(s), a support-platform applied to the deposit-core, a controlled release coating and, an immediate release coating comprising a selective serotonin reuptake inhibitor and one or more pharmaceutically acceptable excipient(s), wherein composition releases more than 15% of selective serotonin reuptake inhibitor in first two hours measured using USP Type II dissolution apparatus in 900 ml of 0.1 N HCl at 50 rpm.

In another embodiment, a stable controlled release pharmaceutical composition comprises a deposit-core comprising a selective serotonin reuptake inhibitor, one or more controlled release agent(s) and one or more pharmaceutically acceptable excipient(s), a support-platform applied to the deposit-core, a controlled release coating and, an immediate release coating comprises about 16% to about 25% of total selective serotonin reuptake inhibitor and one or more pharmaceutically acceptable excipient(s), wherein composition releases more than 15% of selective serotonin reuptake inhibitor in first two hours measured using USP Type II dissolution apparatus in 900 ml of 0.1 N HCl at 50 rpm.

The term ‘pharmaceutically acceptable excipient(s)’ used in pharmaceutical compositions of invention comprise but not limited to diluents, binders, pH stabilizing agents, disintegrants, surfactants, glidants and lubricants.

The amounts of excipient(s) employed will depend upon how much active agent is to be used. One excipient(s) can perform more than one function.

Binders as used in the invention comprises but are not limited to, starches such as potato starch, wheat starch, corn starch; microcrystalline cellulose such as products known under the registered trademarks Avicel, Filtrak, Heweten or Pharmacel; celluloses such as hydroxypropyl cellulose, hydroxyethyl cellulose, hydroxypropylmethyl cellulose (HPMC), ethyl cellulose, sodium carboxy methyl cellulose; natural gums like acacia, alginic acid, guar gum; liquid glucose, dextrin, povidone, syrup, polyethylene oxide, polyvinyl pyrrolidone, poly-N-vinyl amide, polyethylene glycol, gelatin, poly propylene glycol, tragacanth, combinations thereof and other materials known to one of ordinary skill in the art and mixtures thereof.

Fillers or diluents, as used in the invention comprises but not limited to confectioner's sugar, compressible sugar, dextrates, dextrin, dextrose, fructose, lactitol, mannitol, sucrose, starch, lactose, xylitol, sorbitol, talc, microcrystalline cellulose, calcium carbonate, calcium phosphate dibasic or tribasic, calcium sulphate, and the like can be used.

Lubricants as used in the invention comprises but not limited to Mg, Al, Ca or Zn stearate, polyethylene glycol, glyceryl behenate, mineral oil, sodium stearyl fumarate, stearic acid, hydrogenated vegetable oil and talc.

Glidants comprises but not limited to, silicon dioxide; magnesium trisilicate, powdered cellulose, starch, talc and tribasic calcium phosphate, calcium silicate, magnesium silicate, colloidal silicon dioxide, silicon hydrogel and other materials known to one of ordinary skill in the art.

Disintegrants comprises but not limited to starches; clays; celluloses; alginates; gums; cross-linked polymers, e.g., cross-linked polyvinyl pyrrolidone or crospovidone, e.g., POLYPLASDONE XL, cross-linked sodium carboxymethylcellulose or croscarmellose sodium, e.g., AC-DI-SOL from FMC; and cross-linked calcium carboxymethylcellulose; soy polysaccharides; and guar gum. Use of disintegrant according to the invention facilitates in the release of drug in the latter stage and thereby completely releasing the drug from the dosage form.

The pharmaceutical composition may optionally contain a surface-active agent. The preferred agent is copolymers composed of a central hydrophobic chain of polyoxypropylene (poly(propylene oxide)) and polyoxyethylene (poly(ethylene oxide)) that is well known as poloxamer. However, other agents may also be employed such as dioctyl sodium sulfosuccinate (DSS), triethanolamine, sodium lauryl sulphate (SLS), polyoxyethylene sorbitan and poloxalkol derivatives, quaternary ammonium salts or other pharmaceutically acceptable surface-active agents known to one ordinary skilled in the art.

The pharmaceutical composition can be formed by various processes known in the art but not limited to such as by dry granulation, wet granulation, melt granulation, direct compression, double compression, extrusion spheronization, layering and the like. The solvent(s) used in wet granulation include all the solvents well known in the art or the mixtures thereof.

In another embodiment, a pharmaceutical composition of invention comprises one or more controlled release coating.

The term “controlled release coating’ used in pharmaceutical compositions of invention meant any coating wherein release of the active ingredient from pharmaceutical composition is modified to occur at a slower rate than that from an immediate release composition.

Controlled release coating comprises but not limited to extended release coating, enteric coating, partial enteric coating or leaky enteric coating, bioadhesive coating and similar coatings known in the art. These coatings may help the pharmaceutical composition to release the drug at and for the required time.

Controlled release coating comprises controlled release agent(s) selected from hydrophilic or hydrophobic agent(s) or the combinations thereof.

The hydrophobic substance in the coating comprises but not limited to Ammonio methacrylate copolymers type A and B as described in USP, methacrylic acid copolymer type A, B and C as described in USP, Polyacrylate dispersion 30% as described in pH. Eur., Polyvinyl acetate dispersion, ethylcellulose, cellulose acetate, cellulose propionate (lower, medium or higher molecular weight), cellulose acetate propionate, cellulose acetate butyrate, cellulose acetate phthalate, cellulose triacetate, poly(methyl methacrylate), poly(ethyl methacrylate), poly(butyl methacrylate), poly(isobutyl methacrylate), and poly(hexyl methacrylate). Poly(isodecyl methacrylate), poly(lauryl methacrylate), poly(phenyl methacrylate), poly(methyl acrylate), poly(isopropyl acrylate), poly(isobutyl actylate), poly(octadecyl acrylate), waxes such as beeswax, carnauba wax, microcrystalline wax, and ozokerite; fatty alcohols such as cetostearyl alcohol, stearyl alcohol; cetyl alcohol and myristyl alcohol; and fatty acid esters such as glyceryl monostearate, glycerol distearate; glycerol monooleate, acetylated monoglycerides, tristearin, tripalmitin, cetyl esters wax, glyceryl palmitostearate, glyceryl behenate, and hydrogenated castor oil.

The hydrophilic substance in the coating is comprises but not limited to celluloses or their salts or derivatives thereof, hydroxyethylcellulose, hydroxypropyl cellulose, hydroxypropyl methylcellulose (hypromellose), sodium carboxymethyl cellulose, alginic acid or their salts and derivatives thereof, carbomer (Carbopol™), polyethyleneoxide, xanthan gum, guar gum, locust bean gum, poly vinyl acetate, polyvinyl alcohol, lactose, PVA these hydrophilic polymers also act as pore forming agent.

These coating comprises one or more excipients selected from the group comprising coating agents, opacifiers, taste-masking agents, fillers, polishing agents, colouring agents, antitacking agents and the like.

The pharmaceutical composition can be coated by a wide variety of methods. Suitable methods include compression coating, coating in a fluidized bed or a pan and hot melt (extrusion) coating. Such methods are well known to those skilled in the art.

In another embodiment, a process for preparing a controlled release pharmaceutical composition comprises preparing a deposit-core comprising about 75% to about 84% of total selective serotonin reuptake inhibitor with one or more controlled release agent(s) and one or more pharmaceutically acceptable excipient(s); applying a support-platform comprising a polymer, a plasticizer, a binder onto a portion of the surface of the deposit-core by compression to form a tablet; applying a controlled release coating comprising one or more controlled release polymer(s) onto the compressed tablets; preparing an immediate release coating of about 16% to about 25% of total selective serotonin reuptake inhibitor and one or more pharmaceutically acceptable excipient(s), applying the immediate release coating onto the controlled release coating. The immediate release coating may further be coated by suitable coating.

The controlled release pharmaceutical composition of invention comprising Paroxetine can be used to treat and prevent the following disorders: Alcoholism, Anxiety, Depression, Obsessive Compulsive Disorder, Panic Disorder, Chronic Pain, Obesity, Senile Dementia, Migraine, Bulimia, Anorexia, Social Phobia, Pre-Menstrual Syndrome (PMS), Adolescent Depression, Trichotillomania, Dysthymia, Substance Abuse.

The following examples are illustrative of the present invention, and the examples should not be considered as limiting the scope of this invention in any way, as these examples and other equivalents thereof will become apparent to those versed in the art, in the light of the present disclosure, and the accompanying claims.

### BRIEF DESCRIPTION OF DRAWINGS

### X-Ray Diffraction Charts Obtained by Measuring in X-Ray Diffractometer

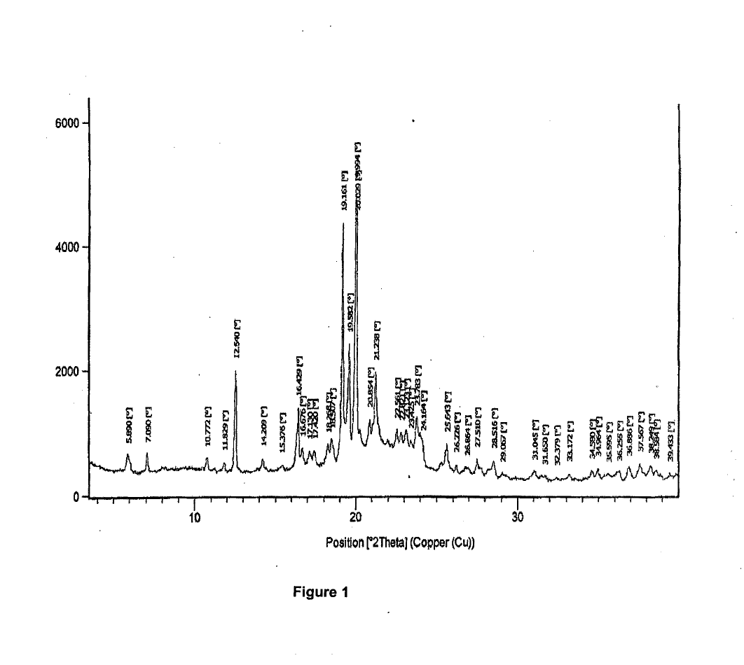

FIG. 1: X-ray diffraction chart of Example 3 of this invention at initial conditions.

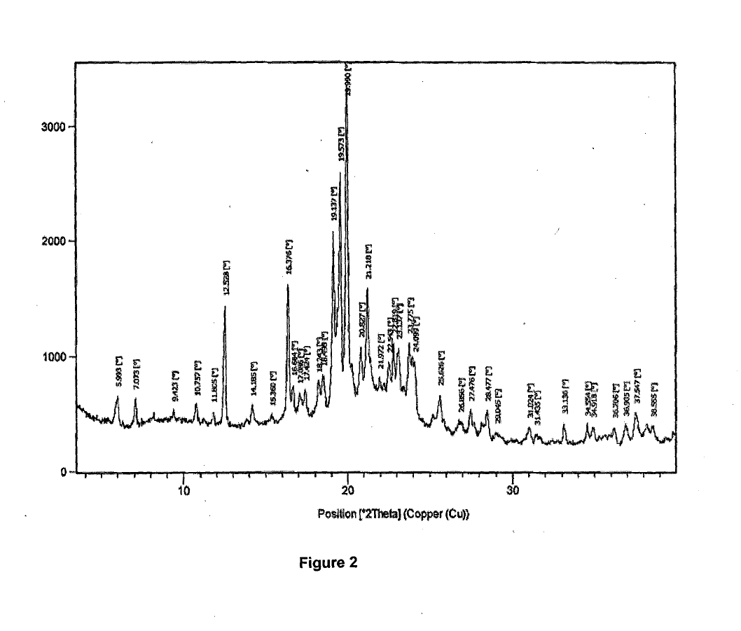

FIG. 2: X-ray diffraction chart of Example 3 after 3 months at 40° C. temperature and 75% RH (Relative Humidity).

### EXAMPLE 1

| | | |
|---|---|---|
| Sr. No. | Ingredients | % w/w |

Part A Deposit-core

| | | |
|---|---|---|
| 1 | Paroxetine HCl Hemihydrate (78%) | 20.26 |
| 2 | Hypromellose | 6.25 |
| 3 | Lactose | 21.62 |
| 4 | Microcrystalline cellulose | 12.50 |
| 5 | Hydroxyethyl cellulose | 12.50 |
| 6 | Xanthan gum | 18.75 |
| 7 | Hydrogenated vegetable oil | 6.25 |
| 8 | Colloidal silicon dioxide | 0.94 |
| 9 | Magnesium Stearate | 0.94 |
| 10 | Methylene chloride | Q.S. |
| | Total | 100.00 |

Part B Support layer

| | | |
|---|---|---|
| 11 | Hydrogenated Vegetable oil | 22.22 |
| 12 | Lactose | 44.44 |
| 13 | Povidone | 11.11 |
| 14 | Hydroxyethyl cellulose | 20.00 |
| 15 | Colloidal silicon dioxide | 1.11 |
| 16 | FD&C Blue | 0.56 |
| 17 | Magnesium Stearate | 0.56 |
| 18 | Isopropyl alcohol | Q.S. |
| | Total | 100.00 |

Part C
1. Enteric coating

| | | |
|---|---|---|
| 19 | Eudragit | 60.29 |
| 20 | Triethyl citrate | 5.00 |
| 21 | Talc | 34.71 |
| 22 | Purified water | Q.S. |
| | Total | 100.00 |

2. Drug loading

| | | |
|---|---|---|
| 23 | Paroxetine HCl Hemihydrate (22%) | 66.09 |
| 24 | Eudragit | 33.91 |
| 25 | Isopropyl alcohol | Q.S. |
| 26 | Acetone | Q.S. |
| | Total | 100.00 |

3. Film coating

| | | |
|---|---|---|
| 27 | Opadry Blue | Q.S. |
| 28 | Purified water | Q.S. |

### Procedure:

### Deposit-Core—

1. Sift Paroxetine HCl Hemihydrate, Hypromellose and Lactose and granulate with methylene chloride.
2. Dried the granules of step 1 and mix with other ingredients of deposit-core.
3. Lubricate step 2 with colloidal silicon dioxide and magnesium stearate.

### Support-Platform—

4. Sift Lactose, Povidone, and Hydroxy ethyl cellulose and granulate using isopropyl alcohol.
5. Dried the granules of step 4 and mix with other ingredients of support-platform.
6. Lubricate step 5 using magnesium stearate.

### Compression—

7. Compress step 3 and step 6 into bilayer tablet.

### Enteric Coating—

8. A dispersion of Eudragit, triethyl citrate and talc in purified water was prepared and coated on to the bilayer tablet of step 7.

### Drug Loading (Immediate Release Coating)—

9. Dissolve Paroxetine HCl Hemihydrate and Eudragit in mixture of isopropyl alcohol and acetone to form solution.
10. Load Drug solution of step 9 on enteric coated bilayer tablets of step 8.
11. Tablets of step 10 optionally coated by film coating solution.

### EXAMPLE 2

| | | |
|---|---|---|
| Sr. No. | Ingredients | % w/w |

Part A Deposit-core

| | | |
|---|---|---|
| 1 | Paroxetine HCl Hemihydrate (83%) | 20.28 |
| 2 | Hypromellose | 5.88 |
| 3 | Lactose | 23.25 |
| 4 | Microcrystalline cellulose | 13.53 |
| 5 | Sodium carboxymethyl cellulose. | 11.76 |
| 6 | Polyethylene oxide | 17.65 |
| 7 | Hydrogenated vegetable oil | 5.88 |
| 8 | Colloidal silicon dioxide | 0.88 |
| 9 | Magnesium Stearate | 0.88 |
| 10 | Purified water | Q.S. |
| | Total | 100.00 |

Part B Support layer

| | | |
|---|---|---|
| 11 | Hydrogenated Vegetable oil | 20.00 |
| 12 | Lactose | 40.00 |
| 13 | Hydroxypropyl cellulose | 10.00 |
| 14 | Hypromellose | 28.00 |
| 15 | Colloidal silicon dioxide | 1.00 |
| 16 | Iron oxide red | 0.50 |
| 17 | Magnesium Stearate | 0.50 |
| 18 | Methylene chloride | Q.S. |
| 19 | Isopropyl alcohol | Q.S. |
| | Total | 100.00 |

Part C
1. Enteric coating

| | | |
|---|---|---|
| 20 | Polyvinyl acetate phthalate | 44.751 |
| 21 | Stearic acid | 3.712 |
| 22 | Diethyl phthalate | 25.769 |
| 23 | Methylene chloride | 25.769 |
| 24 | Methanol | Q.S. |
| | Total | 100 |

2. Drug loading

| | | |
|---|---|---|
| 25 | Paroxetine HCl Hemihydrate (17%) | 58.83 |
| 26 | Ethylcellulose | 41.17 |
| 27 | Isopropyl alcohol | Q.S. |
| 28 | Methylene chloride | Q.S. |
| | Total | 100 |

3. Film coating

| | | |
|---|---|---|
| 29 | Opadry Blue | Q.S. |
| 30 | Purified water | Q.S. |

### Procedure:

### Deposit-Core—

1. Sift Paroxetine HCl Hemihydrate, Hypromellose and Lactose and granulate with purified water.
2. Dried the granules of step 1 and mix with other ingredients of deposit-core.
3. Lubricate step 2 with colloidal silicon dioxide and magnesium stearate.

### Support-Platform—

4. Sift Lactose, Hydroxypropyl cellulose, Hypromellose and Iron oxide red and granulate with methylene chloride.
5. Dried the granules of step 4 and mix with other ingredients of support-platform.
6. Lubricate step 5 using magnesium stearate.

### Compression—

7. Compress step 3 and step 6 into bilayer tablet.

### Enteric Coating—

8. A dispersion of polyvinyl acetate phthalate and diethyl phthalate and stearic acid in a mixture of methanol and methylene chloride was prepared and coated on to the bilayer tablet of step 7.

### Drug Loading (Immediate Release Coating)—

9. Dissolve Paroxetine HCl Hemihydrate and ethyl cellulose in a mixture of isopropyl alcohol and acetone to form solution.
10. Load Drug solution of step 9 on enteric coated bilayer tablets of step 8.
11. Tablets of step 10 optionally coated by film coating solution.

### EXAMPLE 3

| | | |
|---|---|---|
| Sr. No. | Ingredients | % w/w |

Part A Deposit-core

| | | |
|---|---|---|
| 1 | Paroxetine HCl Hemihydrate (80%) | 19.55 |
| 2 | Hypromellose | 5.88 |
| 3 | Lactose | 23.98 |
| 4 | Microcrystalline cellulose | 13.53 |
| 5 | Polyethylene oxide | 11.76 |
| 6 | Sodium alginate | 17.65 |
| 7 | Guar gum | 5.88 |
| 8 | Colloidal silicon dioxide | 0.88 |
| 9 | Magnesium Stearate | 0.88 |
| 10 | Purified water | Q.S. |
| | Total | 100.00 |

Part B Support layer

| | | |
|---|---|---|
| 11 | Hydrogenated Vegetable oil | 20.00 |
| 12 | Lactose | 40.00 |
| 13 | Povidone | 10.00 |
| 14 | Hypromellose | 28.00 |
| 15 | Colloidal silicon dioxide | 1.00 |
| 16 | Iron oxide red | 0.50 |
| 17 | Magnesium Stearate | 0.50 |
| 18 | Purified water | Q.S. |
| | Total | 100.00 |

Part C
1. Enteric coating

| | | |
|---|---|---|
| 19 | Hypromellose acetate succinate | 57.53 |
| 20 | Triethyl citrate | 4.45 |
| 21 | Talc | 38.01 |
| 22 | Purified water | Q.S. |
| | Total | 100.00 |

2. Drug loading

| | | |
|---|---|---|
| 23 | Paroxetine HCl Hemihydrate (20%) | 59.27 |
| 24 | Polyvinyl alcohol | 33.17 |
| 25 | Lecithin | 7.56 |
| 26 | Polyethylene glycol | Q.S. |
| 27 | Purified water | Q.S. |
| | Total | 100.00 |

3. Film coating

| | | |
|---|---|---|
| 28 | Opadry Blue | Q.S. |
| 29 | Purified water | Q.S. |

### Procedure:

### Deposit-Core—

1. Sift Paroxetine HCl Hemihydrate, Hypromellose and Lactose and granulate with purified water.
2. Dried the granules of step 1 and mix with other ingredients of deposit-core.
3. Lubricate step 2 with colloidal silicon dioxide and magnesium stearate.

### Support Layer—

4. Sift Lactose, Povidone, Hypromellose and FD & C Blue and granulate with water.
5. Dried the granules of step 4 and mix with other ingredients of support-platform.
6. Lubricate step 5 using magnesium stearate.

### Compression—

7. Compress step 3 and step 6 into bilayer tablet.

### Enteric Coating—

8. A dispersion of Eudragit and triethyl citrate and talc in water was prepared and coated on to the bilayer tablet of step 7.

### Drug Loading (Immediate Release Coating)—

9. Dissolve Paroxetine HCl Hemihydrate and Opadry in a mixture of isopropyl alcohol and methylene chloride to form solution.
10. Load Drug solution of step 9 on enteric coated bilayer tablets of step 8.
11. Tablets of step 10 optionally coated by film coating solution.

### In-Vitro Dissolution Study:

Table 1 given below shows the comparative dissolution profile of Paroxetine hydrochloride controlled release tablets of Example 3 of the present invention (Test) & Paxil CR® 37.5 mg tablets (Reference) carried out in 900 ml of 0.1 N HCl for 2 hours followed by 900 ml, Phosphate Buffer pH 6.8 as medium using Apparatus USP II (Paddle), at 50 rpm speed. The release profile (cumulative % of drug released) is given in Table 1.

TABLE 1

Cumulative % Drug Release

| Time in | | Paxil CR ® 37.5 mg tablets |
|---|---|---|
| Hours | Example 3 (Test) | (Reference) |
| 0 | 0 | 0 |
| 1 | 17.8 | 0 |
| 2 | 18.5 | 0 |
| 4 | 20.4 | 2.9 |
| 6 | 24.4 | 10.7 |
| 8 | 34.8 | 27 |
| 10 | 46.2 | 48 |
| 12 | 59.3 | 61.4 |
| 14 | 71.8 | 73.2 |
| 16 | 82.9 | 81.5 |
| 18 | 90.5 | 86.5 |

### X-RD Stability Study:

The pharmaceutical composition of Example 3 was kept for stability study at 40° C. temperature and 75% RH (Relative Humidity) conditions for 3 months. The pattern X-ray diffraction for the pharmaceutical composition of Example 3 obtained by measuring in X-ray diffractometer is shown in FIG. 1 and FIG. 2 and found to be stable.

1. A controlled release pharmaceutical composition comprises:
a) a deposit-core comprising a selective serotonin reuptake inhibitor, one or more controlled release agent(s) and one or more pharmaceutically acceptable excipient(s),
b) a support-platform applied to the deposit-core,
c) a controlled release coating and,
d) an immediate release coating comprising a selective serotonin reuptake inhibitor and one or more pharmaceutically acceptable excipient(s).

2. The controlled release pharmaceutical composition of claim 1, wherein selective serotonin reuptake inhibitor is selected from Paroxetine, Citalopram, Escitalopram, Sertraline, Fluoxetine or Desvenlafaxine or pharmaceutically acceptable salts thereof.

3. The controlled release pharmaceutical composition of claim 1, wherein selective serotonin reuptake inhibitor is Paroxetine or pharmaceutically acceptable salts thereof.

4. The controlled release pharmaceutical composition of claim 1, wherein immediate release coating comprises about 16% to about 25% of total selective serotonin reuptake inhibitor and deposit-core comprises about 75% to about 84% of total selective serotonin reuptake inhibitor.

5. The controlled release pharmaceutical composition of claim 1, wherein the controlled release agent(s) in the deposit-core is hydrophilic, hydrophobic or combinations thereof.

6. The controlled release pharmaceutical composition according to claim 5, wherein, the hydrophilic controlled release agent(s) is selected from cellulose derivatives, alginic acid derivatives, polysaccharides, alkylene oxides or mixtures thereof.

7. The controlled release pharmaceutical composition according to claim 5, wherein, the hydrophobic controlled release agent(s) is selected from hydrogenated vegetable oils, polymethacrylates, ethyl cellulose or mixtures thereof.

8. The controlled release pharmaceutical composition of claim 1, wherein, the support-platform comprises a polymeric substance, a plasticizer, a binder and one or more pharmaceutically acceptable excipient(s).

9. A process for preparing a controlled release pharmaceutical composition comprising:
a) preparing a deposit-core comprising about 75% to about 84% of total selective serotonin reuptake inhibitor with one or more controlled release agent(s) and one or more pharmaceutically acceptable excipient(s);
b) applying a support-platform comprising a polymer, a plasticizer and a binder onto a portion of the surface of the deposit-core by compression to form a tablet.
c) applying a controlled release coating comprising one or more controlled release agent(s) onto the compressed tablets of (b) and
d) preparing an immediate release coating of about 16% to about 25% of total selective serotonin reuptake inhibitor and one or more pharmaceutically acceptable excipient(s)
e) applying the immediate release coating onto the controlled release coating.

10. A controlled release pharmaceutical composition of claim 1, wherein composition releases more than 15% of selective serotonin reuptake inhibitor in first two hours measured using USP Type II dissolution apparatus in 900 ml of 0.1 N HCl at 50 rpm.
